(19) United States
(12) Patent Application Publication
(10) Pub. No.: US 2024/0397935 A1
(43) Pub. Date: Dec. 5, 2024

(54) PHOTOSYNTHETIC COMPOSITIONS AND METHODS OF PREPARING AND USING PHOTOSYNTHETIC COMPOSITIONS

(71) Applicant: SYMBIOX, INC., San Diego, CA (US)

(72) Inventors: Jose-Tomás Egaña-Erazo, Santiago (CL); Valentina VELOSO-GIME´NEZ, Santiago (CL); Mauricio P. BORIC, Santiago (CL); Rolando A. REBOLLEDO, Santiago (CL)

(21) Appl. No.: 18/273,974

(22) PCT Filed: Feb. 3, 2022
(86) PCT No.: PCT/US2022/015089

§ 371 Date: Jul. 24, 2023

Related U.S. Application Data
(60) Provisional application No. 63/145,400, filed on Feb. 3, 2021.

(51) Int. Cl. A01N 1/02

(52) U.S. Cl. CPC A01N 1/021

(57) ABSTRACT

Photosynthetic compositions, methods of preparing photosynthetic compositions, and methods of using photosynthetic compositions are provided herein. Some photosynthetic compositions can be perfusable and comprise photosynthetic cells suspended in a biocompatible solution. In some aspects, the photosynthetic cells are present in the composition at a density of between 10⁶-10⁹ cells/ml.

### CROSS-REFERENCE TO RELATED APPLICATIONS

This application claims priority to U.S. Provisional Patent application No. 63/145,400, filed Feb. 3, 2021. All references, including publications, patent applications, and patents, cited herein are hereby incorporated by reference to the same extent as if each reference were individually and specifically indicated to be incorporated by reference and were set forth in its entirety herein.

### FIELD

The disclosure relates to photosynthetic compositions, methods of preparing photosynthetic compositions, and methods of using photosynthetic compositions.

### SUMMARY

Photosynthetic compositions, including perfusable photosynthetic solutions for organ preservation capable of circulating through a tissue or an organ, and photosynthetic solutions for use as blood replacement in conditions where transfusion may be required, are provided herein.

Contemplated perfusable photosynthetic compositions for organ preservation can comprise photosynthetic cells in a biocompatible solution, wherein the photosynthetic cells remain viable for at least 24 hours in the biocompatible solution. In some aspects, the biocompatible solution can be isotonic or nearly isotonic with blood. In some aspects, the biocompatible solution can comprise at least one of a saline solution and a Ringer's lactate solution. In some aspects, the biocompatible solution further comprises a cell impermeant agent (e.g., mannitol). In some aspects, the cell impermeant agent is present in the solution in any suitable concentration, including at a concentration of between 0.1 and 10%, between 0.1 and 5%, or 0.1 and 2% (w/v). In some aspects, the photosynthetic cells are suspended in the biocompatible solution and are present in the composition at any suitable density, including for example, a density of between 10³-10¹² cells/ml, 10⁶-10⁹ cells/ml, or between 10⁶-10⁸ cells/ml. In some aspects, the photosynthetic cells are present in the composition at a density of up to 10⁸, or up to 10⁷ cells/ml. In some aspects, the photosynthetic cells are present in the composition at any suitable density, including for example, a density of at least 10³, 10⁶, at least 10⁷, at least 10⁸, at least 10⁹ cells/ml, or at least at least 10¹⁰ cells/ml. In some aspects, the photosynthetic cells comprise (C. reinhardtii. In some aspects, the photosynthetic cells comprise genetically engineered photosynthetic cells. In some aspects, the biocompatible solution further comprises an oncotic agent (e.g., Dextran-70), which can be present in the biocompatible solution in any suitable concentration, including for example, a concentration of between 0.1 and 25%, between 0.1 and 10%, between 3 and 7%, or between 1 and 10% (w/v). In some aspects, the biocompatible solution further comprises an anticoagulant (e.g., heparin, warfarin), which can be present in the biocompatible solution in any suitable concentration.

In some embodiments, a photosynthetic solution as described herein can be used to preserve organs in static or dynamic systems at different temperatures, including hyponormothermic, normothermic and subnormothermic conditions (e.g., cold preservation on ice after removal and before implantation).

10006| Also provided herein are methods for preparing photosynthetic compositions, and methods for preservation of a tissue or an organ. Contemplated methods can comprise preparing a photosynthetic composition comprising photosynthetic cells in a biocompatible solution, wherein the photosynthetic cells remain viable for at least two weeks, at least one week, at least 72 hours, at least 48 hours, or at least 24 hours in the biocompatible solution. In some aspects, the method can comprise mixing a solution (e.g., at least one of a saline solution and a Ringer's lactate solution) with between 0.1 and 10%, between 0.1 and 5%, or 0.1 and 2% (w/v) of a cell impermeant agent (e.g., mannitol). In some aspects, the method can comprise adding between 0.1 and 25%, between 0.1 and 10%, between 3 and 7%, or between 1 and 10% (w/v) of an oncotic agent (e.g., Dextran-70) to the solution (e.g., at least one of a saline solution and a Ringer's lactate solution, optionally with a cell impermeant agent). In some aspects, the method can comprise suspending photosynthetic cells in the biocompatible solution (which can comprise, for example, one or more of a saline solution, a Ringer's lactate solution, an anticoagulant, an oncotic agent, and a cell impermeant agent) at a cell density of between 10⁵-10⁹ cells/ml, between 10⁶-10⁸ cells/ml, up to 10⁸ cells/ml, or up to 10⁷ cells/ml. In some aspects, the photosynthetic cells are suspended in the biocompatible solution and are present in the composition at a density of between 10³-10¹² cells/ml, or between 10⁶-10⁹ cells/ml. In some aspects, the photosynthetic cells are present in the composition at a density of at least 10⁶, at least 10⁷, at least 10⁸, or at least 10⁷ cells/ml. In some aspects, the photosynthetic cells comprise algal cells, for example, C. reinhardtii. However, all suitable photosynthetic microorganisms are contemplated for the photosynthetic solutions described herein, including, for example, photosynthetic cyanobacteria, or any of the photosynthetic cells as described in any of U.S. Patent Application Publication No. 2016/0058861 and U.S. Pat. Nos. 9,849,150 and 11,207,362, each of which is incorporated herein in its entirety. Exemplary “photosynthetic cells” include cells and cell organisms that are photosynthetically active, for example, photosynthetic cells, cells containing chloroplasts, as well as isolated chloroplasts as long as they release oxygen, including unicellular algae from the genus Chlamydomonoas (e.g., Chlamydomonas reinhardtii ((C. reinhardtii), which can grow and maintain photosynthesis thereby delivering oxygen, which are biocompatible with endothelial cells, can circulate through the vasculature without triggering an in-vivo immune response, can share characteristics with erythrocytes (e.g., diameters, size, complexity, shear-thinning behavior), and can circulate through the vasculature in vivo.). However, it should be appreciated that photosynthetic compositions described herein can comprise any suitable photosynthetic cell(s).

Contemplated methods can additionally or alternatively comprise perfusing the tissue or organ with any of the photosynthetic compositions described herein. In some aspects, the organ is a human organ. In some aspects, the organ is ischemic. In some aspects, the method further comprises illuminating the organ with an illumination device. In some aspects, the method further comprises transplanting the organ into a recipient after perfusing the organ with the photosynthetic composition. In some aspects, perfusing the organ with the photosynthetic composition comprises perfusing the organ ex vivo. In some aspects, perfusing the organ with the photosynthetic composition comprises perfusing the organ in situ. In some aspects, the photosynthetic composition comprises photosynthetic cells in a biocompatible solution, wherein the photosynthetic cells remain viable for at least two weeks, at least one week, at least 72 hours, at least 48 hours, or at least 24 hours in the biocompatible solution. In some aspects, the biocompatible solution comprises a Ringer's lactate solution. In some aspects, the photosynthetic cells are present in the composition at any suitable density, including for example, a density of between 10³-10¹² cells/ml, between 10⁶-10⁹ cells/ml, or between 10⁶-10⁸ cells/ml. In some aspects, the photosynthetic cells are present in the composition at a density of up to 10⁸, or up to 10⁷ cells/ml. In some aspects, the photosynthetic cells are present in the composition at a density of at least 10⁶, at least 10⁷, at least 10⁸, or at least 10⁷ cells/ml. In some aspects, the photosynthetic cells comprise C. reinhardtii.

Other advantages and benefits of the disclosed assemblies, components and methods will be apparent to one of ordinary skill with a review of the following detailed description.

### BRIEF DESCRIPTION OF THE DRAWINGS

The details of embodiments of the present disclosure, both as to their structure and operation, can be gleaned in part by study of the accompanying drawings, in which like reference numerals refer to like parts, and in which:

FIGS. 1A-1C show C. reinhardtii that were incubated in TAP, RLM or a mix of both in a 1:1 ratio in agar plates;

FIGS. 1D-1H show flow cytometry data plots corresponding to C. reinhardtii that were incubated in TAP, RLM or a mix of both in a 1:1 ratio showing viability compared to a death control;

FIGS. 2A-2C show the morphology of C. reinhardtii incubated in TAP, RLM or a mix of both in a 1:1 ratio;

FIGS. 2D-2G show flow cytometry data plots corresponding to cell size of C. reinhardtii incubated in TAP, RLM or a mix of both in a 1:1 ratio;

FIG. 3A is a graph showing O₂ production of photosynthetic solutions with different densities of C. reinhardtii;

FIG. 3B is a graph showing no significant difference in osmolality up to 10⁸ C. reinhardtii/ml;

FIG. 3C is a graph showing no significant difference in rheological properties of the solution up to 10⁸ C. reinhardtii/ml;

10017| FIGS. 3D-3E illustrate normal phenotypes of zebrafish larvae exposed for 24 h to photosynthetic solution containing up to 10⁸ C. reinhardtii/ml compared to a control, and mild and severe mortality observed at 10⁸ and 10⁹ C. reinhardtii/ml, respectively;

FIGS. 4A-4E illustrate the capacity of a photosynthetic solution to produce oxygen to support metabolic requirements of zebrafish larvae and fresh rat kidney slice;

FIGS. 5A-5C illustrate isolated porcine kidneys manually perfused and sliced;

FIGS. 5D-5G show a vascular distribution of the solution in the renal cortex and medulla;

FIGS. 5H-5I show cryosections of perfused kidneys showing distribution of C. reinhardtii in glomeruli and afferent arteriole and medullary blood vessels and capillaries;

FIG. 6A is a schematic representation of an exemplary ex vivo perfusion system;

FIG. 6B-6D illustrate vascular parameters (MAP, perfusion flow, and RVR) measured during photosynthetic perfusion and subsequent flushing;

FIGS. 7A-7B illustrate viability and morphology of microalgae were not affected by perfusion and flushing steps; and

FIGS. 7C-7F show H&E-stained paraffin sections showing normal histological structure of porcine kidneys in cortex and medulla after perfusion.

### DETAILED DESCRIPTION

After reading this description, it will become apparent to one skilled in the art how to practice the claims in various alternative embodiments and alternative applications. However, although various embodiments will be described herein, it is understood that these embodiments are presented by way of example and illustration only, and not limitation. As such, this detailed description of various embodiments should not be construed to limit the scope or breadth of the appended claims.

Oxygen is the key molecule for aerobic metabolism, but no animal cells can produce it, creating an extreme dependency on external supply. In contrast, microalgae are photosynthetic microorganisms, therefore, they are able to produce oxygen as plant cells do. Hypoxia is one of the main issues in organ transplantation, especially during preservation.

The disclosure herein is directed to perfusable photosynthetic solutions that allow organ preservation by in situ vascular oxygenation. The disclosure herein is also directed to photosynthetic solutions suitable for use in, among other things, ex vivo organ preservation, perfusion of organs ex vivo or in situ, or as a blood replacement in conditions such as hemorrhage, where blood transfusion may be required.

In some aspects, perfusable photosynthetic compositions for organ preservation is provided, comprising photosynthetic cells in a biocompatible solution, wherein the photosynthetic cells remain viable for at least 24 hours in the biocompatible solution. In some aspects, the biocompatible solution can be isotonic or nearly isotonic with blood. In some aspects, the biocompatible solution comprises at least one of a saline solution and a Ringer's lactate solution. In some aspects, the biocompatible solution further comprises a cell impermeant agent (e.g., mannitol). In some aspects, the cell impermeant agent is present in the solution in any suitable concentration, including at a concentration of between 0.1 and 10%, between 0.1 and 5%, or 0.1 and 2% (w/v). In some aspects, the photosynthetic cells are present in the composition at a density of between 10⁶-10⁸ cells/ml. In some aspects, the photosynthetic cells are present in the composition at a density of up to 10⁸, or up to 10⁷ cells/ml. In some aspects, the photosynthetic cells are suspended in the biocompatible solution and are present in the composition at a density of between 10⁶-10⁹ cells/ml, or between 10⁶-10⁸ cells/ml. In some aspects, the photosynthetic cells are present in the composition at a density of up to 10⁸, or up to 10⁷ cells/ml. In some aspects, the photosynthetic cells are present in the composition at a density of at least 10⁶, at least 10⁷, at least 10⁸, or at least 10⁷ cells/ml. In some aspects, the photosynthetic cells comprise C. reinhardtii. In some aspects, the photosynthetic cells comprise genetically engineered photosynthetic cells. In some aspects, the photosynthetic cells are genetically modified to improve the solution, for example, to release bioactive molecules, to improve survival, or to improve biocompatibility. In some aspects, contemplated photosynthetic solutions can comprise a combination of wild-type cells and genetically modified photosynthetic cells, for example, those that at least one of improve survival of the cells, improve biocompatibility of the cells, and produce recombinant growth factors or therapeutic agents like anti-inflammatory agents or anti-bacterial agents. In some aspects, the biocompatible solution further comprises an oncotic agent (e.g., Dextran-70), which can be present in the biocompatible solution in any suitable concentration, including for example, a concentration of between 0.1 and 25%, between 0.1 and 10%, between 3 and 7%, or between 1 and 10% (w/v). In some aspects, the biocompatible solution further comprises an anticoagulant (e.g., heparin), which can be present in the biocompatible solution in any suitable concentration. In some aspects, the biocompatible solution can be supplemented with agents that improve oxygen transportation (also known as oxygen carriers) such as hemoderivates (e.g., erythrocytes), perfluorocarbons-based molecules or hemoglobin polymers.

The photosynthetic compositions described herein can be perfusable and suitable for ex vivo perfusion of an organ, for example, for organ preservation during treatment, storage, or transport for transplantation in a recipient or reimplantation in a subject. In some aspects, the photosynthetic composition is biocompatible. In some aspects, the photosynthetic composition comprises a biocompatible solution. In some aspects, the solution has a pH of about 6-8 at room temperature, and an osmolality of about 280-350 mOsm/kg.

In some aspects, the biocompatible solutions can comprise photosynthetic cells as described in any of U.S. Patent Application Publication No. 2016/0058861 and U.S. Pat. Nos. 9,849,150 and 11,207,362, each of which is incorporated herein in its entirety. Exemplary photosynthetic cells include cells and cell organisms that are photosynthetically active, for example, photosynthetic cells as well as isolated chloroplasts as long as they release oxygen, including unicellular algae from the genus Chlamydomonoas (e.g., Chlamydomonas reinhardtii (C. reinhardtii), which can grow and maintain photosynthesis thereby delivering oxygen, which are biocompatible with endothelial cells, can circulate through the vasculature without triggering an in-vivo immune response, can share characteristics with erythrocytes (e.g., diameters, size, complexity, shear-thinning behavior), and can circulate through the vasculature in vivo.). However, it should be appreciated that photosynthetic compositions described herein can comprise any suitable photosynthetic cell(s).

In some aspects, the photosynthetic compositions can comprise photosynthetic cells in a suitable medium, for example, a medium that is isotonic (or nearly isotonic) with blood, such as Ringer's solution or a modified Ringer's solution (for example, lactated Ringer's solution). The disclosed compositions can comprise one or more osmotic agents that increase the osmolality of the composition. Osmotic agents include substances to which capillary walls are impermeable, terms as oncotic agents. Exemplary oncotic agents include albumin, dextran, hydroxyethyl starch, polyethylene glycol (such as PEG-35), and lactobionate. One or more oncotic agents can be used to adjust to the oncotic pressure of a composition to the desired oncotic pressure, which in some embodiments is between 25-30 mm Hg.

The compositions can include additional components, such as one or more reducing agents or buffers. In some embodiments, the composition can include any suitable amount of a reducing agent, such as glutathione, N-acetyl-L-cysteine, or a combination thereof. One of ordinary skill in the art can select additional reducing agents that can be used in the solutions. In some embodiments, the composition can include any suitable amount of a buffer, such as HEPES ((4-(2-hydroxyethyl)-1-piperazineethanesulfonic acid). Additional buffers include, but are not limited to, phosphate (such as sodium phosphate or potassium phosphate), citrate (such as sodium citrate), acetate (such as sodium acetate), or bicarbonate (such as sodium bicarbonate). One of ordinary skill in the art can select additional buffers that can be used in the disclosed solutions. In some embodiments, the composition may include one or more precursors of adenosine triphosphate, such as adenine. In some aspects, additional components, such as antibiotics (for example, penicillin), insulin, and/or dexamethasone can be added prior to using the composition, if desired.

Also disclosed herein are methods for preparing photosynthetic compositions, and methods for preservation of a tissue or an organ. Contemplated methods can comprise preparing a photosynthetic composition comprising photosynthetic cells in a biocompatible solution, wherein the photosynthetic cells remain viable for at least two weeks, at least one week, at least 72 hours, at least 48 hours, or at least 24 hours in the biocompatible solution. In some aspects, the method can comprise adding a cell impermeant agent (e.g., mannitol) to a solution (e.g., at least one of a saline solution and a Ringer's lactate solution) to a concentration of between 0.1 and 10%, between 0.1 and 5%, or 0.1 and 2% (w/v). In some aspects, the method can comprise adding an oncotic agent (e.g., Dextran-70) to the solution (e.g., at least one of a saline solution and a Ringer's lactate solution, optionally with a cell impermeant agent) to a concentration of between 0.1 and 25%, between 0.1 and 10%, between 3 and 7%, or between 1 and 10% (w/v). In some aspects, the method can comprise adding an anticoagulant (e.g., heparin) to the solution (e.g., at least one of a saline solution and a Ringer's lactate solution, optionally with a cell impermeant agent) to any suitable concentration. In some aspects, contemplated methods can comprise adding agents that improve oxygen transportation (also known as oxygen carriers) such as hemoderivates (e.g., erythrocytes), perfluorocarbons-based molecules or hemoglobin polymers, to the solution. In some aspects, the method can comprise suspending photosynthetic cells in the biocompatible solution (which can comprise, for example, one or more of a saline solution, a Ringer's lactate solution, an anticoagulant, an oncotic agent, and a cell impermeant agent) such that the photosynthetic cells are present in the composition at a density of between 10⁵-10⁹ cells/ml, between 10⁶-10⁸ cells/ml, up to 10⁸ cells/ml, or up to 10⁷ cells/ml. In some aspects, the photosynthetic cells comprise algal cells, for example, C. reinhardtii. In some aspects, the photosynthetic cells comprise any suitable photosynthetic microorganisms, including, for example, photosynthetic cyanobacteria, or any of the photosynthetic cells as described in any of U.S. Patent Application Publication No. 2016/0058861 and U.S. Pat. Nos. 9,849,150 and 11,207,362, each of which is incorporated herein in its entirety. Exemplary photosynthetic cells can include cells and cell organisms that are photosynthetically active, for example, photosynthetic cells, cells containing chloroplasts, as well as isolated chloroplasts as long as they release oxygen, including unicellular algae from the genus Chlamydomonoas (e.g., Chlamydomonas reinhardtii (C. reinhardtii), which can grow and maintain photosynthesis thereby delivering oxygen, which are biocompatible with endothelial cells, can circulate through the vasculature without triggering an in-vivo immune response, can share characteristics with erythrocytes (e.g., diameters, size, complexity, shear-thinning behavior), and can circulate through the vasculature in vivo.). However, it should be appreciated that photosynthetic compositions described herein can comprise any suitable photosynthetic cell(s).

10035| Contemplated methods can additionally or alternatively comprise perfusing the tissue or organ, for example, ex vivo or in situ organ perfusion, with any of the photosynthetic compositions described herein. In some aspects, the organ is a human organ. In some aspects, the organ is ischemic. In some aspects, the method further comprises illuminating the organ with an illumination device. In some aspects, the method further comprises transplanting the organ into a recipient after perfusing the organ with the photosynthetic composition. In some aspects, perfusing the organ with the photosynthetic composition comprises perfusing the organ ex vivo. In some aspects, perfusing the organ with the photosynthetic composition comprises perfusing the organ in situ. In some aspects, the photosynthetic composition comprises photosynthetic cells in a biocompatible solution, wherein the photosynthetic cells remain viable for at least two weeks, at least one week, at least 72 hours, at least 48 hours, or at least 24 hours in the biocompatible solution. In some aspects, the biocompatible solution comprises a Ringer's lactate solution. In some aspects, the photosynthetic cells are present in the composition at a density of between 10⁶-10⁹ cells/ml, or between 10⁶-10⁸ cells/ml. In some aspects, the photosynthetic cells are present in the composition at a density of up to 10⁸, or up to 10⁷ cells/ml. In some aspects, the photosynthetic cells are present in the composition at a density of at least 10⁶, at least 10⁷, at least 10⁸, or at least 10⁷ cells/ml. In some aspects, the photosynthetic cells comprise C. reinhardtii.

In some aspects, the organ can be perfused for about 1 hour to 14 days, such as about 1-72 hours, 2-48 hours, 1-48 hours, 1-24 hours, 1-12 hours, 4-24 hours, 1-14 days, 1-10 days, 1-7 days, 2-14 days, 2-10 days, or 5-10 days. In some aspects, the organ can be perfused with a photosynthetic composition that is at any suitable temperatures, including for example, a temperature of between 4-37° C., between 12-37° C., about 20-25° C., or any other suitable temperatures.

The perfusion composition can be delivered to the organ via, among other things, one or more cannulas which are inserted in a vessel of the organ, such as an artery or vein. For example, the perfusion composition can be delivered via one or more cannulas inserted in a vessel that supplies blood (such as oxygenated blood) to an organ. One of ordinary skill in the art can select appropriate vessels for perfusion of an organ. For example, a kidney may be perfused through a cannula inserted in the renal artery, while a liver may be perfused through a cannula inserted in the hepatic artery or a cannula inserted in the portal vein, a heart may be perfused through one or more cannulas inserted in the coronary arteries, and lungs may be perfused through one or more cannulas inserted in the pulmonary arteries. In some embodiments, the flow of the perfusion composition to the organ is a continuous flow, such as a flow without substantial variations of flow rate, for example to mimic venous blood flow under most physiologic conditions. In other embodiments, the flow of the perfusion composition to the organ is a pulsatile flow (such as having flow rate variations that mimic arterial pulsatile blood flow), for example, pulsatile flow of the perfusion composition through a cannula inserted in an artery of the organ. In some aspects, the methods disclosed herein can utilize a dual perfusion technique, where the organ is perfused using pulsatile and continuous flow, for example, simultaneously. For example, some contemplated methods can comprise pulsatile flow perfusion of a liver through the hepatic artery and a continuous flow perfusion of the same liver through the portal vein.

In some examples, the perfusable photosynthetic composition can exit an organ from one or more veins, such as the renal vein, pulmonary veins, hepatic veins, coronary sinus, or vena cava. In some aspects, contemplated methods can include passive venous drainage into a perfusion reservoir. In other aspects, a catheter can be inserted in a vein, for example for selective collection of fluid samples.

Contemplated methods can also comprise administering a photosynthetic composition as described herein into a vein of a mammal. Such methods can comprise administering the composition at any suitable rate (e.g., 5-200 mL/kg/hr, between 5-50 mL/kg/hr, between 1-400 mL/hr, no more than 300 mL/hr). Such methods can comprise administering any suitable volume of the composition (e.g., 1-500 mL, 90-450 mL) for any suitable duration (e.g., between 15 minutes and 3 hours, less than 15 minutes, between 15-60 minutes, less than 1 hour, less than 30 minutes, 1-2 hours, less than 2 hours, at least 15 minutes, at least 30 minutes, at least 1 hour). In some aspects, contemplated methods can comprise administering a photosynthetic composition as described herein, wherein the photosynthetic composition that is at any suitable temperatures, including for example, a temperature of between 4-37° C., between 12-37° C., about 20-25° C., or any other suitable temperatures.

### EXAMPLES

The microalgae Chlamydomonas reinhardtii was incorporated in a standard preservation solution, and key aspects such as alterations in cell size, oxygen production and survival were studied. Osmolarity and rheological features of the photosynthetic solution were comparable to human blood. In terms of functionality, the photosynthetic solution proved to be not harmful and to provide sufficient oxygen to support the metabolic requirement of zebrafish larvae and rat kidney slices. Thereafter, isolated porcine kidneys were perfused, and microalgae reached all renal vasculature, without inducing damage. After perfusion and flushing, no signs of tissue damage were detected, and recovered microalgae survived the process. Thus, Applicant surprisingly discovered the use of photosynthetic microorganisms as vascular oxygen factories to generate and deliver oxygen in isolated organs, representing a novel and promising strategy for organ preservation. However, other uses of the inventive photosynthetic compositions are contemplated, including, for example, as a blood replacement in conditions such as hemorrhage, where blood transfusion is needed.

The lack of appropriate tissue oxygenation represents a major issue in several medical areas, being particularly relevant in the transplantation field, where organ ischemia induces hypoxia, limiting their ex vivo preservation time, as well as their further clinical outcome after transplantation.

Aiming to decrease the oxygen requirements of isolated organs, static cold storage (SCS) has been the gold standard in clinical transplantation settings, reducing metabolism and therefore oxygen consumption in about 90%. However, this technique is limited in terms of preservation time and for maintaining the integrity of suboptimal grafts derived from expanded criteria donor (ECD) and donation after circulatory death (DCD), which are more sensitive to damage. SCS represents the first step in the cascade of ischemia reperfusion injury ensuing organ implantation, triggering tissue damage through oxidative stress and inflammation. Moreover, it is associated with organ allograft dysfunction and acute rejection, reducing the graft survival.

Aiming to provide oxygen ex vivo, novel technologies have been established. Among them, the use of machine perfusion is promising. Some of these systems are based on extracorporeal oxygenation devices, where erythrocytes are oxygenated in a dynamic system that allows them to recirculate through the organ vascular network. Although recent clinical data support their safety and efficacy, the use of blood generates additional complications, because it is not always available on site, has a relative short preservation time and requires the inclusion of a membrane oxygenator, which substantially increases the total cost. Additionally, blood recirculation generates hemolysis, which can be toxic as free hemoglobin can cause inflammation and oxidative stress, thus the development of novel perfusable solutions for ex vivo oxygenation is an active field of research. The use of oxygen carriers as an alternative to erythrocytes has been widely studied, and promising results have been described for hemoglobin-based oxygen carriers obtained from annelids. However, they require intensive purification for their use, and their passive oxygen release kinetics makes them poorly controllable depending on the organ metabolic needs. As a purely synthetic alternative, perfluorocarbons have limitations due to the difficulty of controlling the kinetics of oxygen release, as well as the complexity of manufacturing and the need to incorporate them into different emulsions, limiting their widespread adoption in organ preservation. As an alternative method for oxygen supply, we have proposed that the induction of local photosynthesis could modulate oxygen tension in hypoxic tissues. Based on this, photosynthetic therapies aim to generate a local symbiotic relationship between animal and photosynthetic cells where, in the presence of light, both metabolisms could be coupled with each other. This approach has potential application in several medical fields, including tissue engineering and regeneration, heart ischemia, and tumor treatment.

### Materials and Methods

Microalgae Culture. Cell-wall deficient UVM4-GFP C. reinhardtii strain (cw15-30-derived) was cultured as described previously. See Neupert, J., Karcher, D., and Bock, R. (2009). Generation of Chlamydomonas strains that Efficiently Express Nuclear Transgenes. Plant J. 57, 1140-1150. doi: 10.1111/j.1365-313X.2008.03746.x. Briefly, microalgae were grown photomixotrophically at room temperature (20-25° C.) on either solid Tris Acetate Phosphate (TAP) medium with 1.5% (w/v) agar or in bottles containing different volumes of liquid TAP medium placed in an orbital shaker (180 rpm). For light stimulation, a lamp with the full spectrum of white light was used to provide continuous light exposure (30 μE/m² s). Cell concentration was determined using a Neubauer chamber.

Generation of an exemplary Photosynthetic Solution for Organ Preservation. To prepare a photosynthetic solution for organ preservation (PSOP), Ringer's lactate solution was mixed with 0.5% (w/v) mannitol (RLM; AppliChem Panreac) as impermeant agent (Nicholson and Hosgood, 2017). Liquid cultures of C. reinhardtii in exponential growth phase were grown in 100 ml borosilicate glass bottles up to 5 L depending on the requirements of each experiment. For optimal growth, constant agitation was maintained, either on an orbital shaker or a magnetic stirrer. For harvesting, liquid cultures up to 2 L were centrifuged and 5 L cultures were recovered by decantation; thereafter pelleted algae were resuspended in RLM at different cell densities (10⁶-10⁹ C. reinhardtii/ml). As control groups, TAP medium and a mixed solution of TAP:RLM (in 1:1 ratio) were included. For ex vivo kidney perfusion (see below) and metabolic coupling assay of kidney slices (see below), 5% (w/v) dextran-70 (H979; AK Scientific Inc) was added to RLM to maintain the oncotic pressure.

Microalgae Viability Assays. After 24 h of incubation of C. reinhardtii in RLM, TAP or TAP:RLM, viability of the microalgae was determined by examining growth after 5 days of inoculation in agar plates. As viability probe, microalgae were diluted to 3×10⁵ C. reinhardtii/ml and incubated for 1 h with 25 uM of Fluorescein diacetate (FDA, F1303, Life Technologies). A death control was included by heating C. reinhardtii at 85° C. for 10 min and 10⁵ events per sample were acquired in BD Influx cell sorter (Becton Dickinson). Data was analyzed with FlowJo software (Becton Dickinson) by gating chlorophyll positive cells. In order to discard the contribution of GFP to the FDA signal, unstained, stained, and dead microalgae were used as controls to determine the basal level of fluorescence and set the gate for FDA.

Cell Morphology Evaluation. General morphology of the microalgae in RLM, TAP or TAP:RLM was evaluated by optical microscopy (Leica DM500), as well as by flow cytometry (BD FACSCanto II analyzer, Becton Dickinson). Cell diameter was quantified using 4, 6, 10 and 15 μm size marker beads (F13838; Life Technologies), and 10⁵ events were recorded in the microalgae gate. Data was analyzed with FlowJo software (Becton Dickinson) by gating chlorophyll positive cells.

Oxygen Production of PSOP. After 0 and 24 h of incubation in RLM, the oxygen production of PSOP containing different cell densities (0, 10⁶, 10⁷, 10⁸ and 10⁹ C. reinhardtii/ml) was measured at 28° C. using an Oxygraph System (Hansatech Instruments). Samples (1 ml) were subjected to 10 min of darkness, followed by 10 min of red (455 nm) and blue (630 nm) illumination (8.7 μE/m² s). Oxygen production rate was calculated from the slope of oxygen evolution. Data was normalized and expressed as the oxygen produced by each microalga cell per second.

PSOP Osmolarity and Viscosity. The osmolality of PSOP containing different cell densities (0, 10⁶, 10⁷, 10⁸ and 10⁹ C. reinhardtii/ml) was measured at RT using a cryoscopic osmometer (Osmomat 030, Gonotec). Viscosity was measured in a rheometer, using a 40 mm conical geometry in response to different shear rates (Discovery HR-2, TA Instruments). The gap between the sample and the geometry was set at 300 μm and measurements were carried out at 28° C.

In vivo Toxicity Assay. Zebrafish is a highly characterized and validated model for biomedical toxicity assays, thus ten larvae at 5 days post fertilization (Danio rerio, TAB5 strain) were obtained from our breeding colony and incubated at 28° C., in 12-well plates, and exposed for 24 h to medium (E3, control) or PSOP with increasing cell densities of C. reinhardtii. In order to maintain larvae in optimal conditions, incubations were performed in a 14:10 light-dark photoperiod as we described before (See Alvarez, M., Chávez, M. N., Miranda, M., Aedo, G., Allende, M. L., and Egaña, J. T. (2018). A Novel In Vivo Model to Study Impaired Tissue Regeneration Mediated by Cigarette Smoke. Sci. Rep. 8, 1-12. doi: 10.1038/s41598-018-28687-1), and no additional illumination was provided to induce photosynthetic oxygen production. Then, survival was determined as the percentage of heart beating larvae. For morphological imaging, larvae were anesthetized by immersion in 4.2% (w/v) tricaine (Sigma-Aldrich), euthanized by cold shock (−20° C. for 10 min), fixed in 4% paraformaldehyde and imaged with a stereoscope (Leica S6D).

Metabolic Coupling Assay with Larvae and Rat Kidney Slices. Twenty zebrafish larvae (5 dpf) contained in 1 ml of RLM were added into the electrode chamber of the Oxygraph System (Hansatech Instruments), and the oxygen evolution was measured for 5 min in darkness followed by 5 min of red (455 nm) and blue (630 nm) illumination (8.7 μE/m² s). Then, 200 μL of PSOP containing 10⁸ C. reinhardtii/ml were added (10⁶ C. reinhardtii/larva), and the oxygen evolution was recorded for the next 10 min in the same lighting condition, followed by 10 min of darkness. Oxygen metabolic rate was calculated from the linear slope of oxygen concentration curve. For rat kidney slices the same setting was applied with slight modifications. Male Sprague-Dawley rats (250-400 g) were obtained from the animal facility of INTA, Universidad de Chile (Santiago, Chile). All animal experiments were performed according to protocols approved by the Ethics Committee of Pontificia Universidad Católica de Chile (180813015). Animals were anesthetized with ketamine (90 mg/kg)/xylazine (10 mg/kg) i. p. and the kidneys were washed out of blood by perfusing 5 ml warm RLM solution supplemented with 5% (v/w) dextran-70 through the abdominal aorta with a syringe. The left kidney was excised, cut in half, and mounted in a vibratome to obtain coronal slices. A single central slice (500 μm thick) was incorporated in the oxygraph chamber and incubated with 2 ml of dextran supplemented RLM, and 100 μL containing 2·10⁷ C. reinhardtii were added.

Porcine Kidneys Procurement. Female healthy pigs were selected by weight (35-45 Kg) from a research breeder facility (CICAP-UC Pirque, Santiago, Chile). Animals were sedated with ketamine (25 mg/kg) and midazolam (0.5 mg/kg) and general anesthesia was maintained with 2% isoflurane and animals were connected to mechanical ventilation. Heparin (100-200 UI/kg) was administered to avoid coagulation during organs procurement. Kidneys were isolated and perfused with 500 ml of Custodiol® and kept at 4° C. until experimental studies. Thereafter, pigs were euthanized with thiopental and potassium chloride. All the experiments were performed after the approval of our local ethical committees (approval No. 160126009).

Microalgae Distribution after ex vivo Kidney Perfusion. Porcine kidneys were manually perfused with 50 ml of PSOP (5× 10⁸ C. reinhardtii/ml) and submitted to macroscopic and histological analysis. For low magnification imaging, fresh organs were sliced with a surgical scalpel and pictures were taken using a stereoscope (Leica S6D). Then, biopsies were fixed in 4% paraformaldehyde, included in O.C.T. compound (4,583, Sakura), sliced (30 μm), stained with H&E and imaged with Leica DM500 microscope.

Machine for Dynamic Organ Perfusion. A machine perfusion system was specially designed and manufactured for this study (Sky-Walkers SpA), which consisted of an organ receiving chamber, a volume reservoir, and a centrifugal pump (EC042B IDEA® Motor, Pittman) connected to a reservoir, to carry out the flow to the renal artery. Pressure (TruWave disposable pressure transducers, Edwards Lifesciences) and flow sensors (Biomedicus TX50 Bio-probe flow transducer, Medtronics) were placed in the arterial line and a closed-loop pressure control was designed and manufactured to keep infusion pressure stable within physiological ranges (70-80 mmHg).

Dynamic Sub-normothermic ex vivo Perfusion of Porcine Kidney. Isolated kidneys were cannulated through aortal patch and connected to the perfusion machine described above (Machine for Dynamic Organ Perfusion). Kidneys were perfused at RT for 30 min with continuous recirculating flow, using 1 L of PSOP at 5×10⁷ C. reinhardtii/ml. Then, organs were flushed with RLM containing 5% dextran-70 (w/v) for 40 min, without recirculation. Mean arterial pressure (MAP) and perfusion flow were recorded, and renal vascular resistance (RVR) was calculated as: RVR=MAP/perfusion flow. Samples of PSOP before and during perfusion/flushing were diluted to 3×10⁵ C. reinhardtii/ml, incubated with FDA and analyzed by flow cytometry as described above (Microalgae Viability Assays). After perfusion and flushing, biopsies were fixed in 4% paraformaldehyde, included in paraffin, sliced (4 μm) and stained with H&E.

Statistical Analysis. All assays were performed in at least three independent experiments (unless specified). GraphPad Prism five software (GraphPad Software) was used for statistical analyses. Statistical tests used are described in each result section.

10059| Results

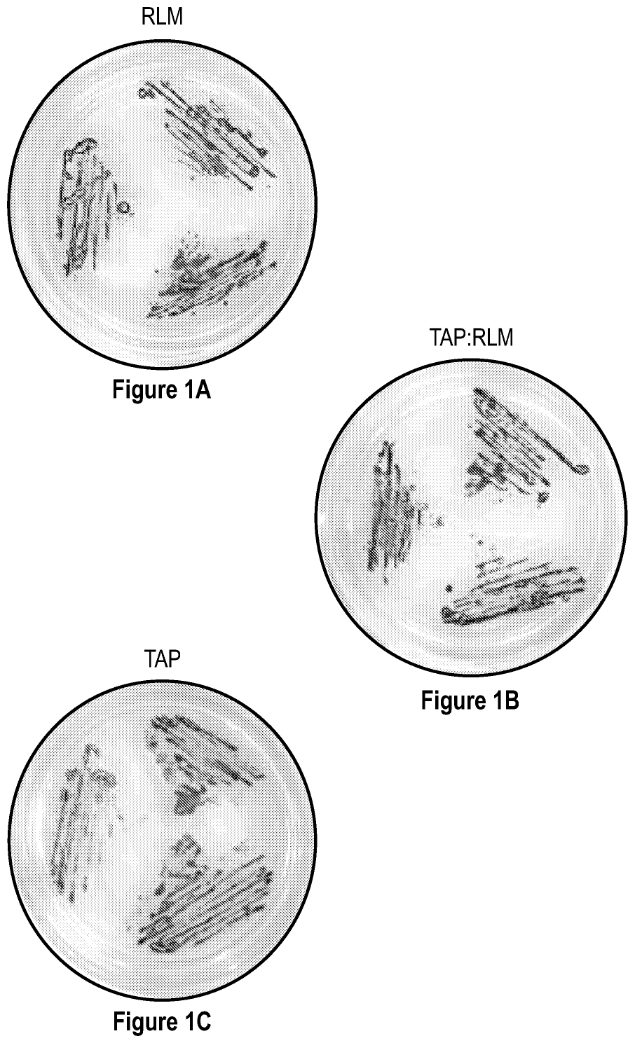

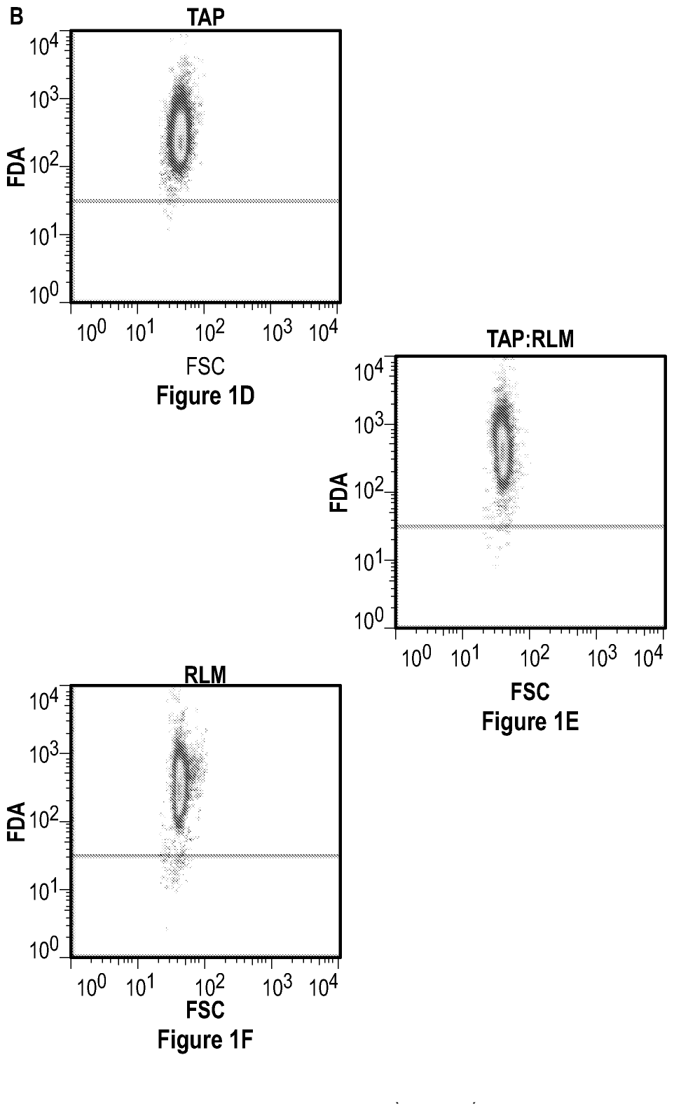

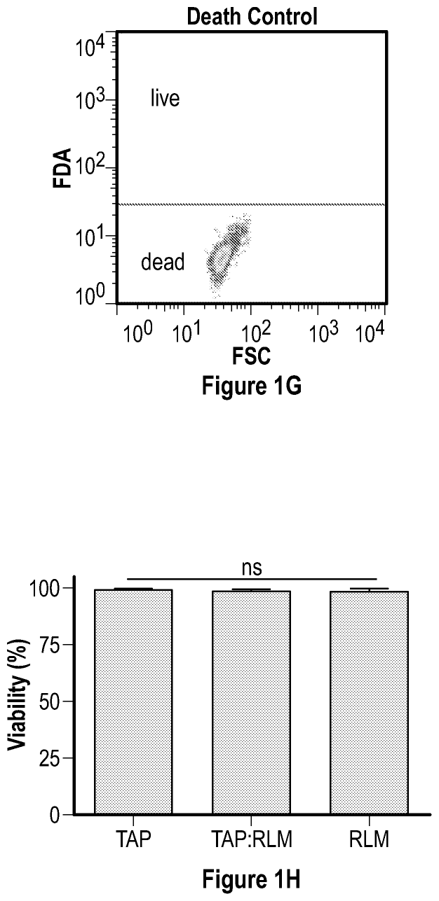

Functional Characterization of PSOP. The first step to study the feasibility of using photosynthetic microalgae in a perfusable solution for ex vivo organ preservation was to evaluate whether the microalgae C. reinhardtii could survive in a standard perfusable physiological solution for organ preservation. Thus, microalgae were incubated for 24 h in RLM, standard algae medium (TAP) or a 1:1 mixture of both. Then, cell viability was assessed, both by evaluating their growing capacity in agar plates (as shown in FIGS. 1A-1C) and by cell cytometry (as shown in FIGS. 1D-1H). Viability of microalgae was not affected as shown by their growing capacity and by flow cytometry. Data are expressed as mean±SD; N=3; ns: non-significant (one-way ANOVA test). No significant differences were observed among groups, confirming that C. reinhardtii remain viable for at least 24 h in RLM.

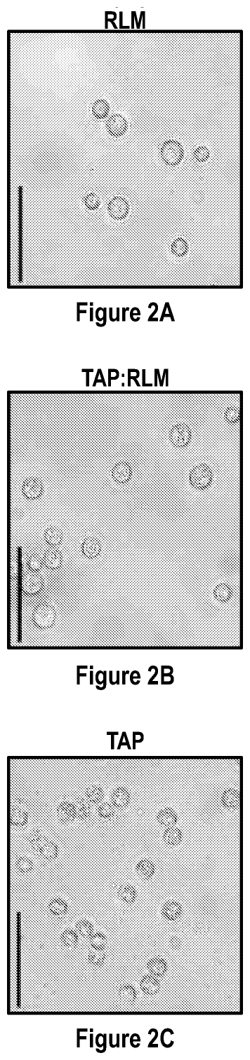

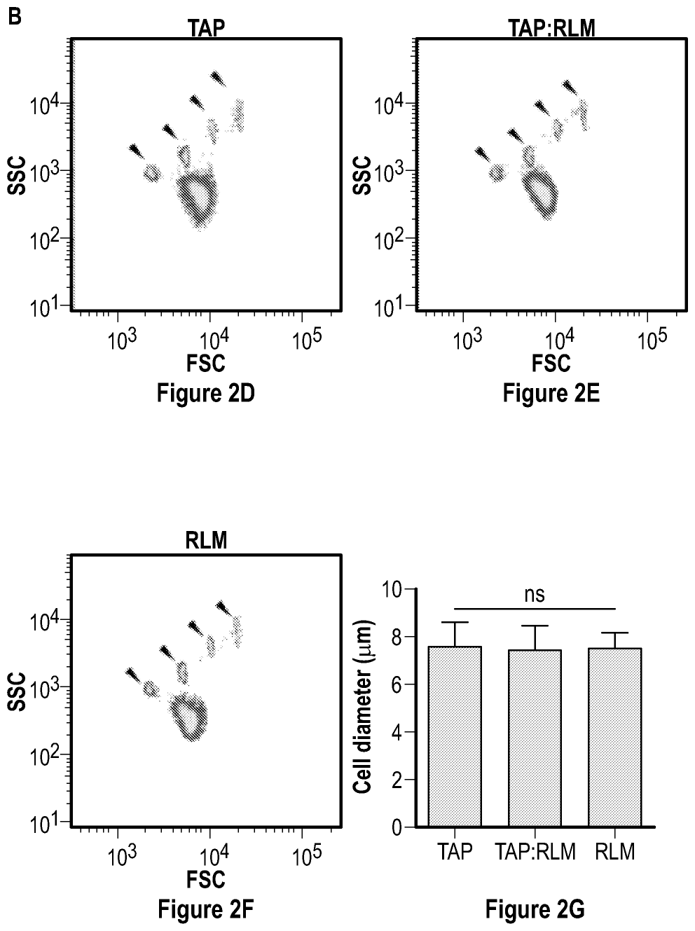

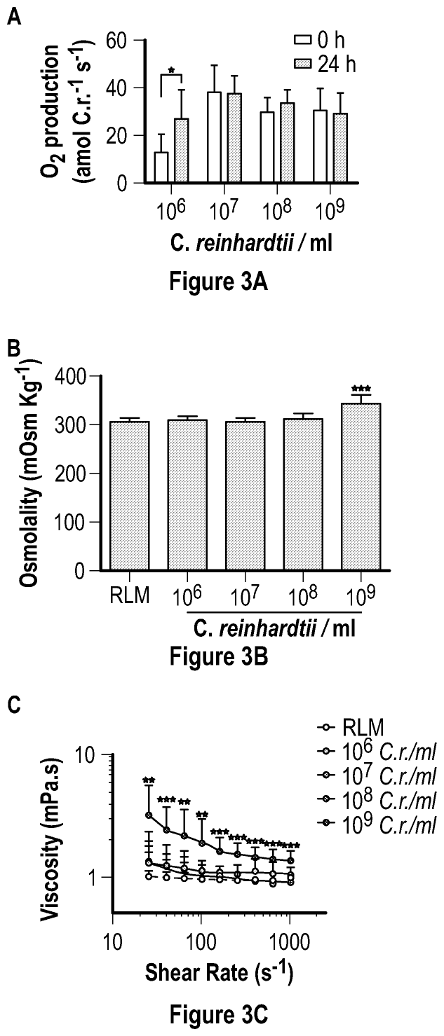

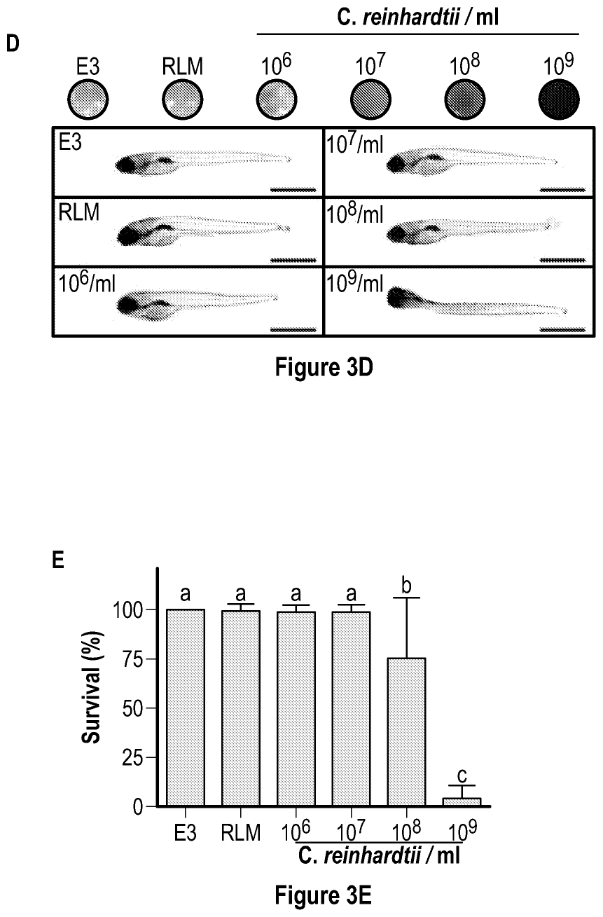

Then, possible morphological changes of C. reinhardtii induced by incubation in the RLM were also evaluated. C. reinhardtii were incubated for 24 h in their culture media (TAP), a standard solution for organ preservation (RLM) or a mix of both in a 1:1 ratio (TAP:RLM). Morphology and size of the microalgae were not affected by the media. Arrow heads indicate size marker beads of 4, 6, 10, and 15 μm in diameter, from left to right. Scale bars represent 25 μm. Data are expressed as mean±SD; N=3; ns: non-significant (one-way ANOVA test). As shown in FIGS. 2A-2C, the general morphology and size of the microalgae did not vary, being further quantified by flow cytometry, where cell diameters of 7.5+0.3, 7.4+0.5 and 7.6+0.4 μm were observed for RLM, TAP:RLM and RLM respectively, as shown in FIGS. 2D-2G.

|0062| Afterwards, oxygen production rates of a photosynthetic solution for organ preservation (PSOP) of RLM containing different densities of C. reinhardtii were characterized and compared. No significant differences in oxygen production were observed between 0 and 24 h of incubation in RLM, nor among the different densities, except for the group containing 10⁶ C. reinhardtii/ml, which presented a higher production rate after 24 h of incubation (as shown in FIG. 3A). Within the studied range, results show that single cell oxygen production was conserved at higher densities, remaining in the 30-40 (amol/cell s) range. Interestingly, no significant differences in cell number were observed after 24 of incubation, suggesting a decrease in the proliferation capacity of the microalgae in this particular experimental setting. Because maintaining osmotic pressure under physiological ranges is a fundamental requirement for perfusable solutions for organ preservation, osmolality was also quantified. As shown in FIG. 3B, the presence of microalgae did not affect the osmolality of the PSOP, remaining in the range of 310 mOsm/Kg, except for 10⁹ C. reinhardtii/ml where the osmolality increased significantly. Up to 10⁸ C. reinhardtii/ml, no significant differences were observed in the osmolality properties of the solution. The effect of the microalgae density in the rheological properties of the PSOP was also studied, measuring viscosity in response to increasing shear rates. Once again, up to densities of 10⁸ C. reinhardtii/ml, viscosity values were comparable to RLM, while at 10⁹ C. reinhardtii/ml, the solution presented significantly higher viscosity (as shown in FIG. 3C).

To evaluate potential toxic effects of the PSOP, zebrafish larvae were used as an in vivo vertebrate model for toxicity testing. Zebrafish larvae were exposed for 24 h to photosynthetic solution containing different densities of microalgae, in the dark. Up to 10⁸ C. reinhardtii/ml larvae presented normal phenotypes compared to control (E3 of FIG. 3D). After incubation, no obvious morphological changes, or signs of damage (such as edema formation and eye size reduction) were observed at different microalgae densities, except for the 10⁹ C. reinhardtii/ml, which induced a general curvature and twisting of the larvae (as shown in FIG. 3D). In terms of viability, solutions containing up to 10⁷ C. reinhardtii/ml were non-toxic for the larvae, while densities of 10⁸ and 10⁹ C. reinhardtii/ml, induced mild and severe mortality respectively (as shown in FIG. 3E). Scale bars represent 1 mm in D. Data are expressed as mean±SD; N=3, 4; * p<0.05, *** p≤0.001 (one-way ANOVA followed by Tukey's test in FIG. 3A; one-way ANOVA followed by Dunnett's test in FIG. 3B; two-way ANOVA followed by Sidak's test in FIG. 3C); different letters in FIG. 3E indicate significant differences with p<0.05 (one-way ANOVA followed by Tukey's test).

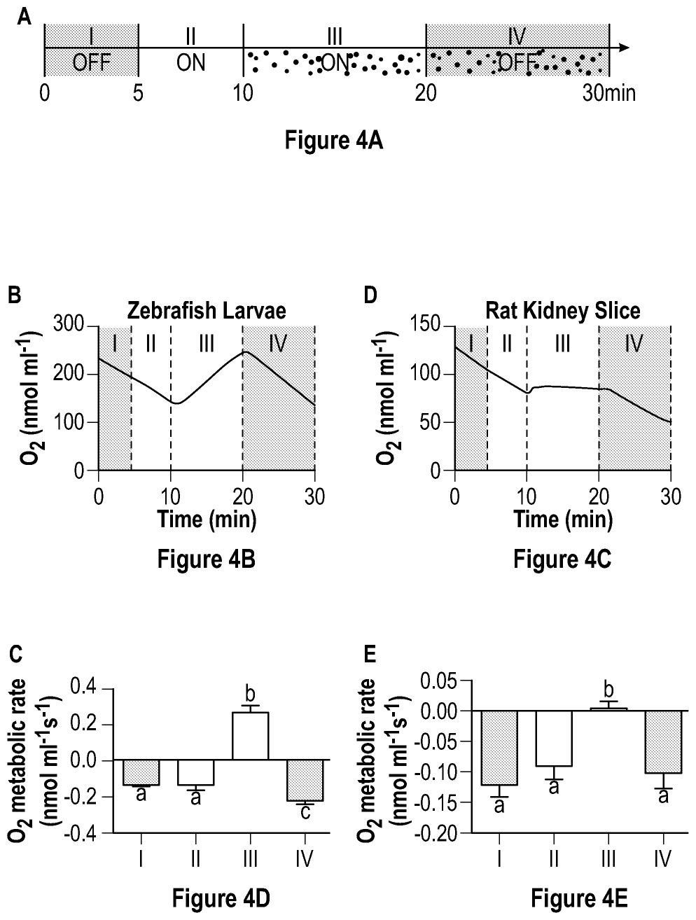

Metabolic Coupling Between PSOP and Animal Systems. The capacity of PSOP to produce enough oxygen to support the metabolic requirements of an active biological system was evaluated. First, zebrafish larvae were placed in an Oxygraph chamber containing RLM, and the oxygen evolution was measured in the absence or presence of microalgae, as well as in the absence or presence of light, as shown in FIG. 4A. Oxygen concentrations were measured for 5 min in darkness (I, OFF) or light (II, ON). Then, microalgae were incorporated, and measurements were performed for 10 min in the presence (III, ON) or absence (IV, OFF) of light (A). In the absence of microalgae, the oxygen concentration decreased overtime, and the negative slope of the curve did not vary in the presence of light, indicating high oxygen consumption of larvae at this stage, as shown in segment I and II of FIGS. 4B and 4C. Afterwards, microalgae were added to the chamber, and the slope of the curve immediately showed a positive slope, meaning that the oxygen production of PSOP exceeded the consumption rate of the larvae, as shown in segment III of FIGS. 4B and 4C. Finally, the light was turned off and a negative slope was observed again, as shown in segment IV of FIGS. 4B and 4C. To validate this data with a more relevant model for transplantation, the same setting was applied to fresh rat kidney slices, and similar results were obtained, as in the presence of light and microalgae, the negative slope of the curve was reverted in segment III, becoming nearly flat, indicating equal oxygen production and consumption; and finally turning negative again in segment IV upon turning off the light, as shown in FIGS. 4D and 4E. A representative curve is shown for each experiment in FIGS. 4B and 4D, and their metabolic rates calculated from the slopes as shown in FIGS. 4C and 4D. Data are expressed as mean±SD; N=3; different letters in FIGS. 4C and 4E indicate significant differences with p<0.05 (one-way ANOVA followed by Tukey's test).

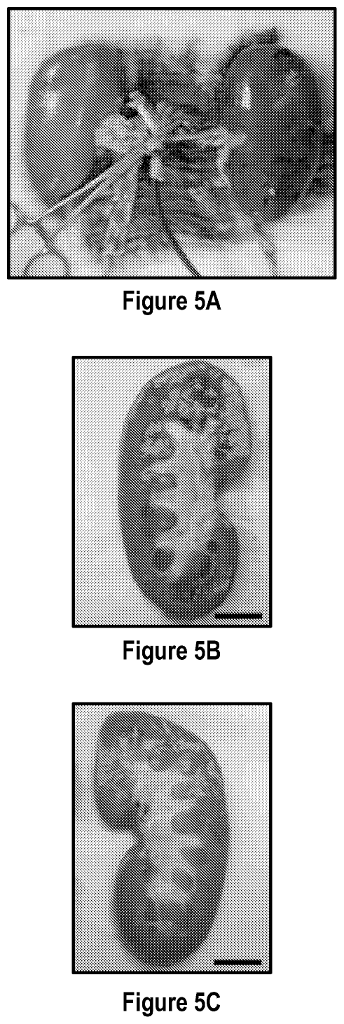

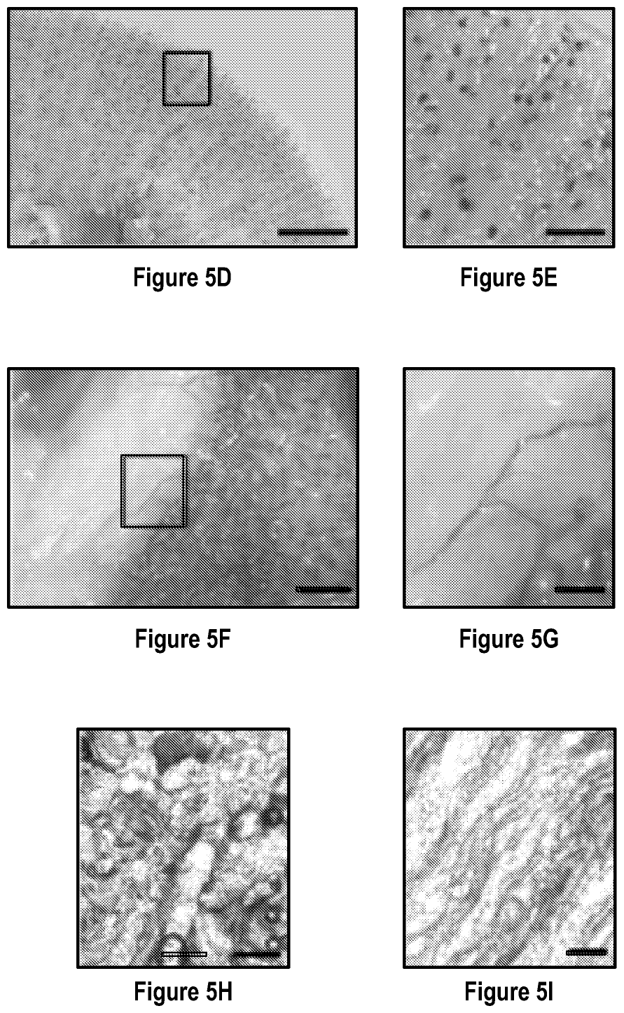

Characterization of ex vivo Perfused Porcine Kidneys. In order to validate the PSOP in a clinically relevant model, isolated porcine kidneys were manually perfused, and renal tissue turned uniformly green, as shown in FIG. 5C and the right side of FIG. 5A, presenting an apparent slight swelling compared to controls shown in FIG. 5B and the left side of FIG. 5A. Besides, no other macroscopic differences were observed among the non-perfused and perfused organs, as shown in FIGS. 5A-5C. Then, fresh perfused tissues were sliced and observed under a stereoscope, clearly showing that microalgae reached the entire vascular territory of the organs, as shown in FIG. 5C. In contrast to calyxes and papilla, where the vascular density is lower, the cortex as well as the medullar pyramid exhibited an intense green color, as shown in FIGS. 5D-5G. Fresh slices show a vascular distribution of the solution in the renal cortex (FIGS. 5D-5E) and medulla (FIGS. 5F-5G). A more detailed localization of the microalgae was microscopically evaluated in cryosections, which showed their homogeneous presence through the entire vascular structures, including the globular distribution in the glomeruli with the afferent arteriole and the characteristic parallel line arrangement of the vessels in the renal medulla, as shown in FIGS. 5H-51. The cryosections of perfused kidneys show the distribution of C. reinhardtii in glomeruli and afferent arteriole (FIG. 5H) and medullar blood vessels and capillaries (FIG. 5I). Scale bar represents 2 cm in FIGS. 5B-5C, 5 mm in FIG. 5D, 1 mm in FIG. 5E and FIG. 5F, 250 μm in FIG. 5G, 100 μm in Figure.

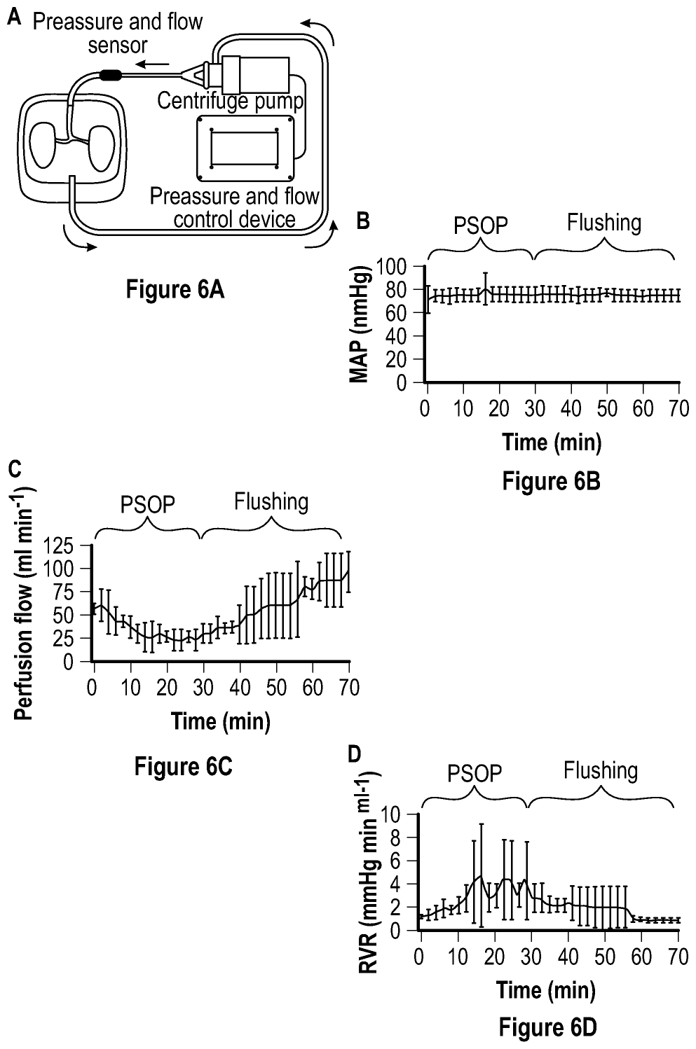

To continue the PSOP validation, perfusion dynamics were evaluated in isolated porcine kidneys, which were connected to an organ perfusion machine prototype specially designed for this study, as shown in FIG. 6A. The perfusion system contains a pressure-flow controlling device, a centrifuge pump, and a container for the isolated organs. This device has automatic flow control based on the sensed values to maintain physiological pressure throughout the procedure. Vascular parameters were measured during the photosynthetic perfusion and the subsequent flushing step. Mean arterial pressure (MAP) was set to 70-80 mmHg remaining stable during the entire procedure. The results showed a stable MAP of 75.5 mmHg during perfusion and the following flushing step, confirming the reliability of the perfusion machine, as shown in FIG. 6B. Perfusion flow decreased during the PSOP perfusion, while renal vascular resistance (RVR) increased, recovering during the flushing step. After the first 5 min, the flow decreased from a mean of 56.7±3.3 to 26.7±8.8 ml/min by 15 min, remaining constant until the flushing step with microalgae-free solution, where the flow gradually recovered and reached values up to 96.7±12.0 ml/min, as shown in FIG. 6C. Accordingly, during PSOP perfusion, calculated RVR increased from 1.26±0.05 to a peak of 4.63±2.49 mmHg min/ml at 16 min, being then less stable with variations between 2.79±0.69 and 4.31±1.89 mmHg min/ml. Thereafter, the initial RVR values were restored after 28 min of flushing, as shown in FIG. 6D. Data are expressed as mean #SEM; N=3 in FIGS. 6B-6D.

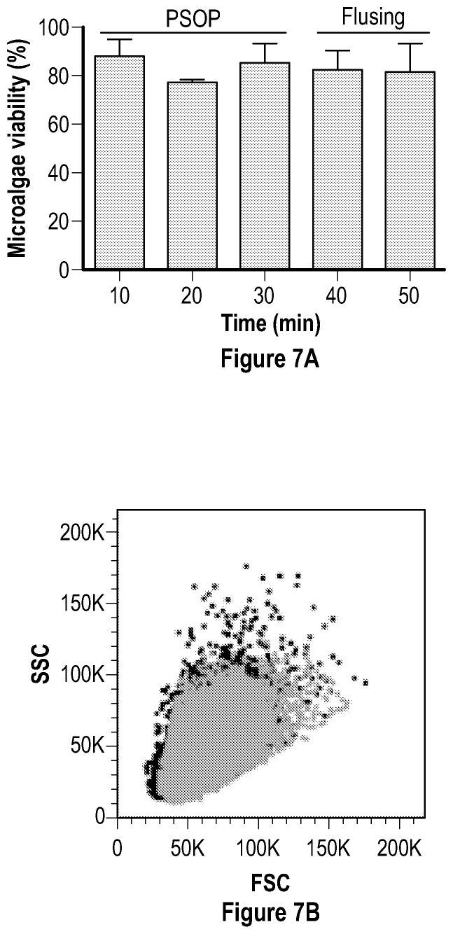

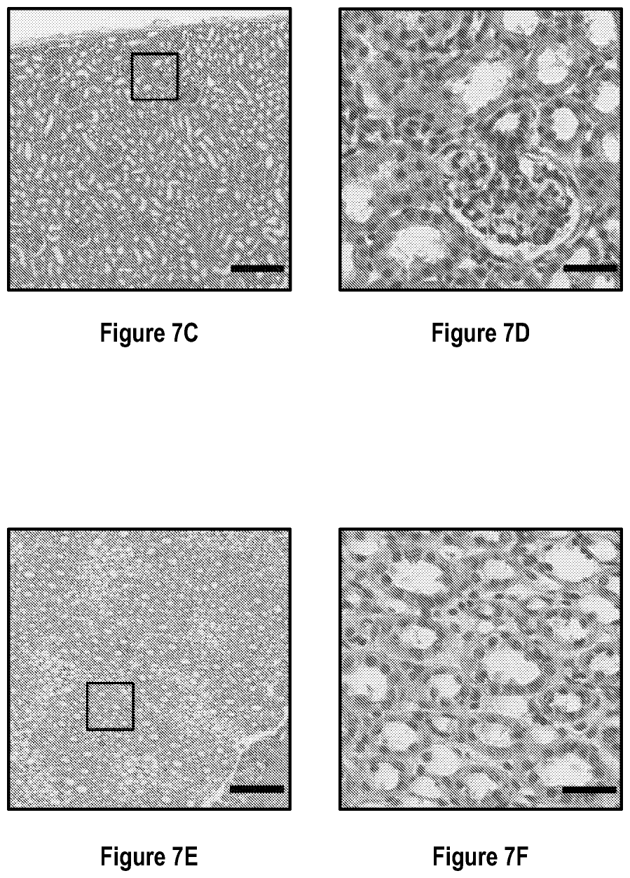

Next, the effect of the perfusion itself in the integrity of the microalgae was studied. Here, samples were collected from the renal vein effluent and analyzed by flow cytometry. Interestingly, as shown in FIG. 7A, the perfusion process did not affect microalgae viability, remaining at about 80% during the perfusion (83.4±6.7%) and the flushing step (81.8±8.5%). In addition to viability, the effect of perfusion in the morphology of the microalgae was also assessed, finding an almost complete overlapping between the microalgae populations obtained before and after perfusion, as shown in FIG. 7B. Finally, tissue integrity was analyzed after perfusion and flushing, and H&E-stained paraffin sections showed the classic kidney architecture/normal histological structure, represented by the cortex, as shown in FIGS. 7C-7D, containing renal corpuscles, proximal and distal tubules, and the medulla with collecting ducts and loops of Henle, as shown in FIGS. 7E-7F.

Overall, results show that glomeruli and tubules did not present signs of damage, as neither necrotic cells in Bowman's capsule nor blood cells or microalgae in Bowman's space were detected. The black and grey dots in FIG. 7B indicate microalgae samples of the solution obtained before and after 10 min of perfusion, respectively, showing an almost complete overlapping of the signal where gray dots masked the microalgae population represented by black dots. Scale bar represents 200 μm (FIGS. 7C and 7E) and 30 μm (FIGS. 7D and 7F). Data are expressed as mean±SD; N=2 in FIGS. 7A-7B.

### Discussion

With this study, the feasibility to incorporate photosynthetic microorganisms in organ perfusion solutions to provide an alternative intravascular source of oxygen to isolated organs is demonstrated. This concept could potentially generate a new physiological state of normoxic ischemia, where the lack of blood supply may not necessarily trigger hypoxia. The use of photosynthetic microorganisms as local oxygen factories may have significant advantages compared to the standard approaches. Among others, in some aspects there is no need for additional carriers, and the local oxygen release kinetics could be easily controlled by the light intensity provided. Additionally, this approach can allow the generation of genetically modified photosynthetic organisms that, in addition to oxygen, could locally release fresh bioactive recombinant molecules as well.

Ringer's lactate solution was chosen as the base to develop the PSOP because of its extended clinical use as physiological fluid. Mannitol was added as an impermeant agent to prevent cell swelling, one of the main requirements for preservation solutions. For kidney perfusion and oxygraphy of kidney slices, dextran-70 was also added to maintain the oncotic pressure and prevent tissue edema. Although cell proliferation was not observed after 24 h of incubation in either medium (RLM and TAP), Ringer's lactate and mannitol solution (RLM) showed high biocompatibility with C. reinhardtii, without affecting microalgae viability and morphology, nor their photosynthetic capacity overtime. This result is surprising because preservation solutions have to meet specific physicochemical requirements that differ from the optimal culture conditions of the microalgae. For instance, the osmolality of the microalgae medium (TAP) is around 64 mOsm/Kg while it is 305 mOsm/Kg for RLM, and previous studies have shown how an increase in osmotic stress can negatively affect cell growth and photosynthetic rates in C. reinhardtii. With exception of the highest microalgae density, all tested PSOP presented the same flowing behavior and dynamic viscosity values as RLM. It is worth noting that in all groups viscosity tended to decrease with higher shear rates (shear thinning), resembling blood behavior and exhibiting appropriate rheological properties for organ perfusion. Moreover, at most concentrations of microalgae, the photosynthetic solution was biocompatible with zebrafish larvae. This toxicity model was chosen because it is extensively used in several fields of research and has been widely validated as a reliable model to test toxicity for biomedical application. In fact, the sensibility of this model shows that both, morphology and viability were significantly affected by incubations in PSOP at the highest density of microalgae, allowing to better define the potential clinical range for a safe microalgae perfusion procedure. Without intending to be bound by any particular hypothesis, the toxic effects at high microalgae densities could be due to several reasons including a high oxygen consumption of the microalgae under such insufficient illumination conditions, or due to issues related to the increased viscosity and osmolality of the PSOP.

Appropriate illumination devices would be beneficial to provide adequate illumination needed for optimal inner organ illumination to evaluate the functional effect of the PSOP in the oxygenation and further preservation of isolated organs. Here, the oxygenation capacity of PSOP in zebrafish larvae was evaluated. In addition to the advantages described herein, in contrast to other organisms, larvae are fully permeable, thus their entire gas interchange occurs by diffusion, allowing to quantify the metabolic interaction between the photosynthetic oxygen produced by the PSOP and the living animal tissues. Considering that five dpf larvae mass are approximately 0.5 mg each, it follows that 10⁹ microalgae suspended in RLM would be sufficient to oxygenate 1 gram of tissue. However, because larval stages are highly hypermetabolic, this number of microalgae might be overestimated. Oxygen requirements of human cells can widely vary ranging from values below 1 to 350 amol/cells. For example, human liver cells in culture are described to consume around 100 amol/cells, which is promising when compared to the results showing that each microalga in the PSOP solution is capable to produce 30-40 amol/cells upon light exposure.

Therefore, a ratio of 3:1 microalgae to cell would be sufficient to ensure optimal tissue oxygenation for that cell type, especially relevant when considering that hepatocytes are roughly 500 times larger in volume and the metabolic oxygen requirements of tissues decrease by 50% at sub-normothermic conditions. In fact, the results herein shows that 2×10⁶ microalgae were sufficient to match the oxygen consumption of a 500 μm-thick rat kidney slice, weighting approximately 45 mg. The setting described above strongly resembles the famous experiment performed by Joseph Priestley in 1772 where he showed that, when placed in a close compartment, a plant can provide enough oxygen to supply the metabolic requirements of a mouse.

It should be appreciated that the composition of the PSOP described here can be modified and optimized according to each particular clinical application, including its chemical composition, photosynthetic strain, and illumination setting.

As kidney represent the most frequent organ transplanted worldwide, the perfusion dynamic of PSOP in isolated porcine kidneys was initially evaluated. Additionally, due to the kidney's intrinsic complexity, this approach allows to test the solution under highly challenging conditions, providing important information about its ex vivo rheological properties, and the effect of the perfusable solution in the different renal vascular domains. Interestingly, C. reinhardtii could reach even the smallest capillaries of porcine kidneys and survive the circulation process without damaging the general tissue architecture of the organs. However, a transient increase in the vascular resistance to flow was observed during 5×10⁷ cell/ml PSOP perfusion, which while not intending to be bound by any particular hypothesis, can be due mostly to a simple increase in fluid viscosity as inferred from the rheological characterization, as shown in FIG. 3C. This analysis, together with the recovery of resistance upon flushing, minimized the probability that reversible vascular occlusion could occur in this setting.

While the examples herein are directed to C. reinhardtii compositions, it should be appreciated that the photosynthetic compositions described herein could comprise any suitable photosynthetic microorganism(s) depending on, for example, the organ preservation settings. For instance, cell size may be a consideration. Photosynthetic microorganisms range widely in size, from Ostreococcus tauri with less than 1 μm of diameter, being the smallest free-living eukaryote known. Optimal temperature may be another consideration. Some species such as Entemoneis kufferati and Synechococcus lividus live in environments from 0 up to 72 Celsius degrees, respectively. Other key features can include, among other things, osmolality, oxygen production, or illumination requirements.

Non-limiting embodiments of the disclosure are provided below.

Embodiment 1. A perfusable photosynthetic composition for organ preservation, comprising: photosynthetic cells in a biocompatible solution, wherein the photosynthetic cells remain viable for at least 24 hours in the biocompatible solution.

Embodiment 2. A perfusable photosynthetic composition of embodiment 1, wherein the biocompatible solution is isotonic or nearly isotonic with blood.

Embodiment 3. A perfusable photosynthetic composition of any of embodiments 1-2, wherein the biocompatible solution comprises at least one of a saline solution and a Ringer's lactate solution.

Embodiment 4. A perfusable photosynthetic composition of any of embodiments 1-3, wherein the biocompatible solution further comprises a cell impermeant agent.

Embodiment 5. A perfusable photosynthetic composition of any of embodiments 1-4, wherein the cell impermeant agent is mannitol, and wherein the mannitol is present at a concentration of between 0.1 and 2% (w/v).

Embodiment 6. A perfusable photosynthetic composition of any of embodiments 1-5, wherein the photosynthetic cells are present in the composition at a density of between 10⁶-10⁹ cells/ml.

Embodiment 7. A perfusable photosynthetic composition of any of embodiments 1-6, wherein the photosynthetic cells are present in the composition at a density of up to 10⁷ cells/ml.

Embodiment 8. A perfusable photosynthetic composition of any of embodiments 1-7, wherein the photosynthetic cells comprise C. reinhardtii.

Embodiment 9. A perfusable photosynthetic composition of any of embodiments 1-8, wherein the photosynthetic cells comprise genetically engineered photosynthetic cells.

Embodiment 10. A perfusable photosynthetic composition of any of embodiments 1-9, wherein the biocompatible solution further comprises at least one of an oncotic agent and an anticoagulant.

Embodiment 11. A perfusable photosynthetic composition of any of embodiments 1-10, wherein the at least one of the anticoagulant and the oncotic agent is Dextran-70, and wherein the Dextran-70 is present in a concentration of between 1-10% (w/v).

Embodiment 12. A method for preservation of an organ, comprising: preparing a photosynthetic composition comprising photosynthetic cells in a biocompatible solution, wherein the photosynthetic cells remain viable for at least 24 hours in the biocompatible solution; and perfusing the organ with the photosynthetic composition.

Embodiment 13. A method of embodiment 12, wherein the organ is a human organ.

Embodiment 14. A method of any of embodiments 12-13, wherein the organ is ischemic.

Embodiment 15. A method of any of embodiments 12-14, further comprising transplanting the organ into a recipient after perfusing the organ with the photosynthetic composition.

Embodiment 16. A method of any of embodiments 12-15, further comprising illuminating the organ with an illumination device.

Embodiment 17. A method of any of embodiments 12-16, wherein perfusing the organ with the photosynthetic composition comprises perfusing the organ ex vivo.

Embodiment 18. A method of any of embodiments 12-17, wherein perfusing the organ with the photosynthetic composition comprises perfusing the organ in situ.

Embodiment 19. A method of any of embodiments 12-18, wherein the biocompatible solution comprises a Ringer's lactate solution.

Embodiment 20. A method of any of embodiments 12-19, wherein the photosynthetic cells are present in the composition at a density of between 10⁶-10⁹ cells/ml.

Embodiment 21. A method of any of embodiments 12-20, wherein the photosynthetic cells comprise C. reinhardtii.

Thus, specific photosynthetic compositions and related methods have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

As used herein, and unless the context dictates otherwise, the term “coupled to” is intended to include both direct coupling (in which two elements that are coupled to each other contact each other) and indirect coupling (in which at least one additional element is located between the two elements).

The recitation of ranges of values herein is merely intended to serve as a shorthand method of referring individually to each separate value falling within the range, and including the endpoints. Unless otherwise indicated herein, each individual value is incorporated into the specification as if it were individually recited herein.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “assembly,” “mechanism,” “element,” “device,” and the like may not be a substitute for the word “means.” As such, no claim element is to be construed as a means plus function unless the element is expressly recited using the phrase “means for.”

Various modifications to the embodiments described herein will be readily apparent to those skilled in the art, and the general principles described herein can be applied to other embodiments without departing from the spirit or scope of the claims. Thus, it is understood that the scope of the claims fully encompasses other embodiments that may become obvious to those skilled in the art and that the scope of the claims is accordingly not limited.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A perfusable photosynthetic composition for organ preservation, comprising:
photosynthetic cells in a biocompatible solution, wherein the photosynthetic cells remain viable for at least 24 hours in the biocompatible solution.

2. The perfusable photosynthetic composition of claim 1, wherein the biocompatible solution is isotonic or nearly isotonic with blood.

3. The perfusable photosynthetic composition of claim 1, wherein the biocompatible solution comprises at least one of a saline solution and a Ringer's lactate solution.

4. The perfusable photosynthetic composition of claim 1, wherein the biocompatible solution further comprises a cell impermeant agent.

5. The perfusable photosynthetic composition of claim 4, wherein the cell impermeant agent is mannitol, and wherein the mannitol is present at a concentration of between 0.1 and 2% (w/v).

6. The perfusable photosynthetic composition of claim 1, wherein the photosynthetic cells are present in the composition at a density of between 10⁶-10⁸ cells/ml.

7. The perfusable photosynthetic composition of claim 1, wherein the photosynthetic cells are present in the composition at a density of up to 10⁷ cells/ml.

8. The perfusable photosynthetic composition of claim 1, wherein the photosynthetic cells comprise C. reinhardtii.

9. The perfusable photosynthetic composition of claim 1, wherein the photosynthetic cells comprise genetically engineered photosynthetic cells.

10. The perfusable photosynthetic composition of claim 1, wherein the biocompatible solution further comprises an oncotic agent.

11. The perfusable photosynthetic composition of claim 10, wherein the oncotic agent is Dextran-70, and wherein the Dextran-70 is present in a concentration of between 1-10% (w/v).

12. A method for preservation of an organ, comprising:
preparing a photosynthetic composition comprising photosynthetic cells in a biocompatible solution, wherein the photosynthetic cells remain viable for at least 24 hours in the biocompatible solution; and
perfusing the organ with the photosynthetic composition.

13. The method of claim 12, wherein the organ is a human organ.

14. The method of claim 12, wherein the organ is ischemic.

15. The method of claim 12, further comprising transplanting the organ into a recipient after perfusing the organ with the photosynthetic composition.

16. The method of claim 12, further comprising illuminating the organ with an illumination device.

17. The method of claim 12, wherein perfusing the organ with the photosynthetic composition comprises perfusing the organ ex vivo.

18. The method of claim 12, wherein perfusing the organ with the photosynthetic composition comprises perfusing the organ in situ.

19. The method of claim 12, wherein the biocompatible solution comprises a Ringer's lactate solution.

20. The method of claim 12, wherein the photosynthetic cells are present in the composition at a density of between 10⁶-10⁸ cells/ml.

21. The method of claim 12, wherein the photosynthetic cells comprise C. reinhardtii.
